(19) United States
(12) Patent Application Publication
(10) Pub. No.: US 2016/0074069 A1
(43) Pub. Date: Mar. 17, 2016

(54) METHODS OF SELECTION OF MAMMALIAN OOCYTES

(71) Applicant: Victoria Link Limited, Wellington (NZ)

(72) Inventors: Kenneth Pattrick McNatty, Wellington (NZ); Janet Louise Pitman, Wellington (NZ)

(73) Assignee: Victoria Link Limited, Wellington (NZ)

(21) Appl. No.: 14/485,079

(22) Filed: Sep. 12, 2014

(51) Int. Cl. A61B 17/435; C12Q 1/68

(52) U.S. Cl. CPC A61B 17/435; C12Q 1/6888; C12Q 2600/158; C12Q 2600/16

(57) ABSTRACT

The invention provides methods of selection of mammalian oocytes. In particular, the invention provides in-vitro methods of selection of viable oocytes from a population of mammalian oocytes by ranking and selecting viable oocytes.

### TECHNICAL FIELD

The present invention relates generally to methods of selection of mammalian oocytes. In particular, the present invention relates to methods of selection of mammalian oocytes by ranking and selecting viable oocytes.

### BACKGROUND ART

The aim of every assisted reproduction treatment is to enhance the likelihood of a pregnancy and the birth of a healthy infant (Land and Evers, 2003).

Known methods of selection of good quality oocytes includes morphological evaluation of the cumulus-oocyte complex (such as the location of the meiotic spindle with respect to the polar body position; Ng et al., 1999; Rattanachaiyanont et al., 1999; Lee et al., 2001; Moffatt et al., 2002) and metabolomic profiling of the oocyte culture media (Nel-Themaat and Nagy, 2011). Although significant increases in pregnancy rates following in vitro fertilisation (IVF) have been achieved (Gardner and Sakkas, 2003) the accuracy of predicting which oocytes will develop to a live birth is still only approximately 10%.

Recent evidence suggests that the expression of some candidate genes in cumulus cells (CC) have the potential to serve as markers of oocyte quality (McKenzie et al., 2004; Gebhardt et al., 2011; Wathlet et al., 2011; Fragouli et al., 2012; Lager et al., 2012). However there is a need for a reliable and accurate method for selecting from a pool of oocytes which has the best potential to develop into a viable blastocyst (after in vitro fertilisation and embryo development and subsequently a healthy baby following implantation and live birth).

### PRIOR REFERENCES

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications may be referred to herein; this reference does not constitute an admission that any of these documents form part of the common general knowledge in the art, in New Zealand or in any other country.

### OBJECTS OF THE INVENTION

It is an object of the invention to provide improved methods of selection of mammalian oocytes that that addresses at least some of the problems of the prior art, such as those discussed above.

Alternatively, it is an object of the present invention to address the foregoing problems or at least to provide the public with a useful choice.

### DISCLOSURE OF INVENTION

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

According to one aspect of the present invention there is provided an in-vitro method of selection of viable oocytes from a population of mammalian oocytes from an individual mammal comprising the steps:
a) measuring the level of expression of at least one cumulus cell gene in cumulus cells associated with each of the population of mammalian oocytes as a marker for oocyte quality;
b) ranking each oocyte from the population of mammalian oocytes of an individual mammal by its measured gene expression from step a); and
   selecting a subset of viable mammalian oocytes that have the best ranked gene expression from step b) for subsequent in vitro fertilisation.

The term “mammalian” as used herein refers to human mammals and non-human mammals such as rats, cows and sheep. The inventors consider that given the similarities in oocytes and reproductive techniques between humans and other non-human mammals, there is reasonable scientific prediction that the method of the present invention can be applied to non-human mammals.

The term “expression” as used herein broadly refers to the process by which DNA is converted by transcription into messenger RNA (mRNA) for subsequent translation into a protein.

The term “gene” as used herein refers to a nucleic acid molecule comprising an ordered series of nucleotides that encodes a gene product (i.e. specific protein).

The term “best ranked” as used herein refers to the highest or lowest expression of a single cumulus cell gene or combined expression of more than one cumulus cell gene, or highest or lowest weighted single cumulus cell gene or combined expression of more than one cumulus cell gene.

Preferably, the measurement step a) is carried out at a ‘MII’ stage of oocyte development.

For the purposes of this specification, the ‘MII’ stage of oocyte development means the stage of development between “MI” oocyte and “2PN” fertilised embryo.

Preferably, the mammalian oocytes are human oocytes.

Preferably, the at least one cumulus cell gene is between 4 and 32 cumulus cell genes.

More preferably, the at least one cumulus cell gene is 4 cumulus cell genes.

More preferably, the step b) is ranking each oocyte from the population of mammalian oocytes by its combined measured gene expression and step c) is selecting a subset of viable mammalian oocytes that have the best combined ranked gene expression.

Preferably, the at least one cumulus cell gene is a follicular maturation process gene.

Preferably, the cumulus cell gene is selected from the group comprising: versican (VCAN); follicle-stimulating hormone receptor (FSHR); hyaluronan synthase 2 (HAS2); progesterone receptor (PR); neuropilin 1 (NRP1); activated leukocyte cell adhesion molecule (ALCAM).

Preferably, the level of expression of the at least one cumulus cell genes in step a) is measured by quantitative Polymerase Chain Reaction (qPCR).

More preferably, the qPCR is multiplex qPCR.

More preferably, the levels of expression of the at least one oocyte quality marker genes in step a) as measured by quantitative Polymerase Chain Reaction (qPCR) is completed within 8 to 24 hours.

The term “polymerase chain reaction or PCR” as used herein refers to a system for in vitro amplification of DNA. Two synthetic oligonucleotide primers, which are complementary to two regions of the target DNA (one for each strand) to be amplified, are added to the target DNA (that need not be pure), in the presence of excess deoxynucleotides and Taq polymerase, a heat-stable DNA polymerase. In a series of temperature cycles, the target DNA is repeatedly denatured, annealed to the primers (typically at 50-60° C.) and a daughter strand extended from the primers. As the daughter strands themselves act as templates for subsequent cycles, DNA fragments matching both primers are amplified exponentially.

The detection of the amplified nucleic acid may be by any of a wide range of techniques known to those skilled in the art, including but not limited to size separation techniques such as gel electrophoresis, probe detection systems either on solid supports or in solution and DNA microarray techniques.

Preferably, the subset of viable mammalian oocytes selected in step c) is three.

According to second aspect of the present invention there is provided an in-vitro method of assisted reproduction comprising the steps:
a) measuring the level of expression of at least one cumulus cell gene in the cumulus cells associated with each of the population of mammalian oocytes from an individual mammal as a marker for oocyte quality;
b) ranking each oocyte from the population of mammalian oocytes by its measured gene expression from step a); and
c) selecting a subset of viable mammalian oocytes that have the best ranked gene expression from step b) for subsequent in vitro fertilisation.

According to a third aspect of the present invention there is provided a kitset for carrying out an in-vitro method of selection of viable oocytes from a population of mammalian oocytes from an individual mammal, the kitset comprising at least one pair of PCR primers configured to bind to a cumulus cell gene, the method comprising the steps:
a) measuring the level of expression of at least one cumulus cell gene in the cumulus cells associated with each of the population of mammalian oocytes as a marker for oocyte quality;
b) ranking each oocyte from the population of mammalian oocytes by its measured gene expression from step a); and
c) selecting a subset of viable mammalian oocytes that have the best ranked gene expression from step b) for subsequent in vitro fertilisation.

The term “primers” as used herein are short nucleic acids, preferably DNA oligonucleotides 15 nucleotides or more in length, which are annealed to a complementary target DNA strand by nucleic acid hybridization to form a hybrid between the primer and the target DNA strand, then extended along the target DNA strand by a polymerase, preferably a DNA polymerase. Primer pairs can be used for amplification of a nucleic acid sequence, e.g. by the polymerase chain reaction (PCR) or other nucleic acid amplification methods well known in the art. PCR-primer pairs can be derived from the sequence of a nucleic acid according to the present invention, for example, by using computer programs intended for that purpose such as Primer (Version 0.5© 1991, Whitehead Institute for Biomedical Research, Cambridge, Mass.).

According to a fourth aspect of the present invention there is provided a method of detecting potentially viable oocytes from a population of mammalian oocytes from an individual comprising the step of measuring the level of expression of at least one cumulus cell gene in the cumulus cells associated with each of the population of mammalian oocytes as a marker for oocyte quality.

### BRIEF DESCRIPTION OF FIGURES

The invention will now be described by way of example only and with reference to any one of the accompanying drawings in which:

FIG. 1 shows linear regressions of log 2 CC number versus C_{T} value for the housekeeping genes RPL19 and 185. Comparisons were made of log 2 CC number against 185 using SYBR Green QPCR (closed black squares) and RPL19 using TaqMan QPCR (open squares).

FIG. 2 shows biplots of six target genes (HAS2), FSHR, ALCAM, VCAN, PR and NRP1) along the first and second principal components (PC1 and PC2). Dots represent each MII oocyte (n=227) from the 25 women. In this analysis PC1 accounted for 50.3%, whilst PC2 accounted for 17.4% of the observed variance. The closer the dots, the more similar the gene expression pattern is for individual CC masses according to the two principal components. The closer the arrows to each other, the more the gene expressions (normalized values) are correlated.

FIG. 3 Rankings of individual MII oocytes according to CC gene expression for patient #24. Individual MII oocytes are spread along the X-axis for each CC candidate gene (HAS2, FSHR, ALCAM, VCAN, NRP1 and PR) from left to right according to their numerical order assigned at the time of oocyte collection. Expression results for each gene are separated by grey vertical lines. Green ‘+’ marks are associated with dots (MII oocytes) that resulted in SET and live birth, whilst black ‘+’ marks are associated with dots (MII oocytes) that progressed to an additional good quality blastocyst that was frozen. Different dots denote different ranks ( [symbol] =1, [symbol] =2, [symbol] =3, [symbol] =4, [symbol] =5, [symbol] =6). Gene expression is presented in 2^{-ΔCT} values (HAS2: 2000×2^{-ΔCT}, FSHR: 10 000×2^{-ΔCT}, ALCAM: 5000×2^{-ΔCT}, VCAN: 100×2^{-ΔCT}, NRP1: 500×2^{-ΔCT} and, PR: 10 000×2^{-ΔCT}).

FIG. 4 The combined rankings of CC expressed genes for individual MII oocytes from 25 patients (P1-P25) according to mRNA levels of (A) HAS2 and FSHR or (B) HAS2, FSHR, VCAN and PR. The green circles represent those MII oocytes that progressed to SET and resulted in live birth, the blue circles represent those MII oocytes that progressed to good quality blastocysts, whilst the red circles represent MII oocytes associated with a negative outcome. The rankings are presented in proportions. To calculate the combined ranking value for individual MII oocytes within each patient (1) the rankings for individual MII oocytes for each CC gene was converted to proportions, by dividing the ranking number by the total number of MII oocytes for a patient and (2) the ranking (in proportions) of each parameter was added and divided by the number of parameters taken into consideration for each individual MII oocyte.

### SUMMARY OF THE INVENTION

The key question being addressed with the present invention is whether the ranking of MII oocytes, at the oocyte stage in frozen oocyte cycles and the 2PN stage (18 h after sperm microinjection) in fresh ICSI cycles, according to a cohort of cumulus expressed genes can provide a reliable method of choosing oocytes with good blastocyst development and live birth potential.

To assess the efficiency of selecting good quality oocytes from the total number of MII oocytes available, retrospective results of ranking MII oocytes based on CC mRNA levels were compared with random selection of MII oocytes and with results obtained following the use of all MII oocytes for treatment. The CC-derived candidates genes, as potential oocyte quality markers, were selected based on results from previous studies of human CC (McKenzie et al., 2004; Cillo et al., 2007; Assidi et al., 2011; Gebhardt et al., 2011; Wathlet et al., 2011) and on the molecular pathways in the COC that are activated during the penultimate phases of folliculogenesis (Otsuka et al., 2005; Hernandez-Gonzalez et al., 2006; Robker et al., 2009). The genes selected were: hyaluronan synthase 2 (HAS2) which is critical for the formation and expansion of the CC mass and expression levels correlate with early embryological development (McKenzie et al., 2004; Cillo et al., 2007); follicle-stimulating hormone receptor (FSHR) for which expression levels are regulated by oocyte secreted factors (Otsuka et al., 2005); solute carrier 2, member 4 (SLC2A4, also known as GLUT4) which is associated with energy metabolism in the COC and is one of the glucose transporters in CC (Roberts et al., 2004); versican (VCAN) which is a major component of the COC extracellular matrix and has been reported as one of the most promising oocyte quality markers in CC (Adriaenssens et al., 2010; Gebhardt et al., 2011; Wathlet et al., 2011); activated leukocyte cell adhesion molecule (ALCAM) which mediates homophilic (ALCAM-ALCAM) and heterophilic (ALCAM-CD6) cell-to-cell interactions, is expressed in CC and is also involved in the initial step of human embryo implantation (Fujiwara et al., 2003; Adriaenssens et al., 2010; Wathlet et al., 2011); secreted frizzled-related protein 2 (SFRP2) which is a soluble modulator of Wnt signalling, and is expressed in CC of mice around the time of ovulation (Hernandez-Gonzalez et al., 2006); progesterone receptor (PR) which is expressed in the dominant follicle of women and its expression in CC was recently reported to be related to oocyte competence (Robker et al., 2009; Wathlet et al., 2013) and neuropilin 1 (NRP1) which is a membrane-bound co-receptor to tyrosine kinase receptor for vascular endothelial growth factor and members of the semaphorin family proteins (Assidi et al., 2011), and up-regulation of NRP1 expression in CC may indicate an oocyte with a positive pregnancy outcome (Assidi et al., 2011). The present invention presents the validation of a multiplex quantitative polymerase chain reaction (QPCR) method capable of measuring up to four genes simultaneously and nine genes in total from extracts of individual CC masses.

### DETAILED DESCRIPTION OF THE INVENTION INCLUDING BEST MODES

Materials and Methods

Patient History and Hormonal Stimulation for IVF

A total of 28 women provided informed consent to participate. All women underwent IVF treatment with single embryo replacement (SET) at Fertility Associates, Wellington clinic [www.fertilityassociates.co.nz (28 Aug. 2013, date last accessed)] and were investigated in relation to embryological and pregnancy outcomes. All women were <38 years old (mean age of 32.1 years) at the time of oocyte collection, and exhibited normal basal plasma FSH levels (<9 IU/l), regular 26-32-day menstrual cycles and were in their first or second IVF cycle. Similarly, the women recruited had a BMI of <28, exhibited no signs of androgenicity, PCOS or endometriosis, were free of any chronic diseases and were not on any form of medication. The causes for infertility were mainly of male origin, although in some cases there was evidence for fallopian tube pathology. Whilst taking the oral contraceptive pill (OCP), they underwent ovarian down-regulation with a GnRH agonist (buserelin, Suprafact; Sanofi-Aventis, Paris, France), followed by ovarian hyperstimulation using daily injections of FSH (150 IU; Puregon, Merck & Co./MSD, NJ, USA) (Damario et al., 1997).

CC-Oocyte Complex (COC) Collection and Embryology

A total of 291 individual CC masses were recovered. From these, 21 individual CC masses from three women were used for the validation of the multiplex TaqMan QPCR, including the validation of the housekeeping gene RPL19. The remaining 270 individual CC masses from 25 women were investigated for potential molecular markers in relation to oocyte developmental indicators (blastocyst and live birth outcome). At 36 h after the administration of an ovulation trigger (i.e. 250 IU of hCG, Ovidrel; Merck Serono, Geneva, Switzerland), COC were collected, rinsed in G-MOPS Plus media (Vitrolife, Goteborg, Sweden) and transferred to individual culture wells of a 4-well plate (Nalge Nunc International, Rochester, N.Y., USA) containing 0.5 ml G-IVF Plus (Vitrolife) culture media under paraffin oil (OVOIL; Vitrolife). At 38 h post-hCG treatment, COC were exposed to 0.5 ml (10 IU) of hyaluronidase solution (HYASE-10× in G-Mops Plus media, Vitrolife) for several seconds before being transferred to 0.5 ml G-MOPS Plus media where CCs were mechanically dissociated from the oocyte. Each denuded oocyte was then transferred to a new dish (BD Falcon, NJ, USA) in individual 15 μl culture drops (G-IVF Plus) under paraffin oil (OVOIL). For each CC sample, both residual solutions (G-MOPS Plus media and HYASE solution) containing the dissociated CC mass for each individual oocyte were pooled in a 1.5 ml tube (#2230-00; SSI, Lodi, Calif. 95240, USA) and centrifuged at 900 g for 1 min. The supernatant was removed and each pellet containing CC was snap frozen in liquid nitrogen for total RNA extraction. Viability of CC from individual COC (N=59) was assessed previously by embryologists working at Fertility Associates after denudation and centrifugation of CC of some patients (N=11). CC viability was determined using a 0.4% Trypan Blue Stain solution (Ajax Finechem Pty Ltd, Auckland, New Zealand) and was found to be 86.4±1.3% (mean±SEM; unpublished data). At 39 h post-hCG treatment, all metaphase II oocytes were subjected to ICSI and cultured individually in 15 μl micro-drops of sequential culture media (G5 Series, Vitrolife) under paraffin oil (OVOIL) in 378 C incubators (MINC; Cook, Brisbane, Australia) under 5% O₂ and 6% CO₂ according to manufacturer's protocols. Embryo transfer was carried out in Embryo Glue media (Vitrolife) using standard protocols. All manipulations (except ICSI) were performed with the aid of a stereomicroscope fitted with a heated stage set at 37° C. The ICSI procedure was performed using an inverted microscope fitted with a heated stage set at 37° C.

The morphological appearances of oocytes and embryos were recorded from the day of COC collection (Day 0) until Day 6 of embryo culture. Following the removal of CC, oocytes and embryos were graded using an inverted microscope. Oocytes were classified according to meiotic stage, i.e. GV, MI, MII or abnormal (giant and/or atretic oocytes). Embryos were assessed at 18, 25, 42, 66, 114 and 138 h post-ICSI using common grading systems (Cummins et al., 1986; Sakkas et al., 1998; Gardner and Sakkas, 2003) for fertilization, early cleavage, cleavage (Day 2), assessment (Day 3), blastocyst development (Day 5) and the final assessment of the in vitro culture outcome (Day 6), respectively. All additional good quality embryos (3BB or better) were frozen in individual straws using Vitrolife freezing media. Pregnancy was determined from plasma hCG levels at Day 14 after embryo transfer and the presence of a foetal heart beat (FHB) by ultrasound scan at 8 weeks after embryo transfer. The recording of pregnancies and live births was carried out according to Fertility Associate's and the Australia and New Zealand Assisted Reproduction Database (ANZARD) requirements [http://www.npsu.unsw.edu.au/data-collection/australian-newzealand-assisted-reproduction-database-anzard (28 Aug. 2013, date last accessed)]. A summary of the embryological outcomes from the 270 COC recovered from the 25 women are presented in Table I.

TABLE I
Summary of the indicators of oocyte
development in the 25 women recruited in this study.

| Indicators | Unit | Outcome |
|---|---|---|
| Oocyte (Day 0) | | |
| Genomic maturity (MII) | % (#) | 85.9 (232/270) |
| Embryo (Day 1-6) | | |
| Fertilization (2PN) | % (#) | 80.6 (187/232) |
| Early stage (≧7 cell) | % (#) | 60.3 (140/232) |
| Blastocyst (3BB or better) | % (#) | 24.1 (56/232) |
| Pregnancy | | |
| hCG (Day 14, ≧18 IU/L) | % (#) | 84.0 (21/25) |
| FHB (Week 8) | % (#) | 76.0 (19/25) |
| Live birth | % (#) | 76.0 (19/25) |

FHB, fetal heart beat.

RNA Extraction and cDNA Synthesis

Total RNA was extracted from each CC mass using the ArrayPure™ Nanoscale RNA Purification Kit that includes a genomic DNA removal step (Epicentre Biotechnologies, Madison, Wis., USA) and cDNA was synthesized in a total volume of 20 ml using SuperScript® VILO™ cDNA Synthesis Kit (Invitrogen, Carlsbad, Calif., USA) according to the manufacturer's protocols. Samples of cDNA were stored at −80° C. for Taqman QPCR analyses.

Quantitative PCR

All primers and Taqman probes (Table II) were designed using the computer package ‘Beacon Designer’ (Premier Biosoft International, Palo Alto, Calif., USA) and manufactured by Invitrogen and Sigma-Proligo (Taqman probes; Proligo-France SAS 1, Paris, France or Proligo-Singapore Pte Ltd, Helios, Singapore), respectively.

TABLE II
Nucleotide (nt) sequences for primers and TaqMan probes for QPCR including
the size of the resultant amplicon with regard to the number of nt for
human ALCAM (NM_001627.3), SFRP2 (NM_003013.2), NRPI (NM_003873.5), PR
(NM_001202474.1), VCAN (NM_004385.4), HAS2 (NM_005328), FSHR (NM_000145),
GLUT4 (NM_001042), RPL19 (NM_000981) and 18S (NR_003286) mRNA.

| Gene | Taqman probe (5′-3′) | Primers (5′-3′) | Size (nt) |
|---|---|---|---|
| ALCAM | (HEX)TTCCTGCCGTCTGCTCTTCTGCC | F-CACCAAGAAGGAGGAGGAATATG | 149 |
| | TCTT (BHQ1) | R-GCAAGGTATGATAATGGTATCTCCA | |
| SFRP2 | (ROX)AAACAACAAACAACAACCAACCA | F-GCCCACCCGAATCTTGTAGAAA | 173 |
| | GACCCAAGT (BHQ2) | R-CAACCTCAGTGGGAAGTGAAAATC | |
| NRPI | (ROX)CATCTTCCAGTCCGAGCCGTTGT | F-GCACCTCATTCCTACATCA | 183 |
| | TG (BHQ2) | R-CTTGCGTTTGCTGTCATC | |
| PR | (HEX)CACTTTCTTCTCCCATGCTTTAT | F-GTGGTCAATAGTGTTTGCTG | 193 |
| | CTCCATCAAT (BHQ1) | R-GTAGGAAGCAAAGGTGGAA | |
| VCAN | (6FAM)TCCCATTCGCAGCCTTTAGCAT | F-ATGGCTTTTCCTGGACAG | 130 |
| | CAT (BHQ1) | R-GGAGTCCTTAGTTCCATCAG | |
| HAS2 | (HEX)AACTGCCCGCCACCGACCCTCC | F-GGTCGTCTCAAATTCATCTGATCTC | 145 |
| | (BHQ1) | R-GGATACATAGAAACCTCTCACAATGC | |
| FSHR | (6FAM)TCTGCTGTAGCTGGACTCATT | F-AAGTTGATTATATGACTCAGGCTAGG | 100 |
| | GTCTTCTGCC (BHQ1) | R-AACTCAGTGTACGTCATGTCAAATC | |
| GLUT4 | (ROX)CCCGCCCTCGCACGTCACTCCG | F-TCTCCGGGTCCTTGGCTTG | 137 |
| | (BHQ2) | R-GCGAAGATGAAAGAACCGATCC | |
| RPLI9 | (CY3)CCAATGCCAACTCCCGTCAGCA | F-GACCCCAATGAGACCAATGAAATC | 105 |
| | GATCCG (BHQ3) | R-GGAATGGACCGTCACAGGCTTG | |
| 18S | | F-GCCGCTAGAGGTGAAATTCTTG | 128 |
| | | R-CGGAACTACGACGGTATCTGATC | |

F, forward; R, reverse.

For validation of the housekeeping gene, 60S ribosomal protein L19 (RPL19), the gene expression level of RPL19 in individual CC masses was compared with that of an alternative housekeeping gene, ribosomal RNA 185, and to the CC number. A total of 21 individual CC masses from 3 women were collected and re-suspended in 50 μl of G-MOPS Plus media (Vitrolife). From each suspension, an aliquot of 20 μl was used for determining total cell number per CC mass. From the remaining, 30 μl of cell suspension, total RNA was extracted and cDNA was synthesized using methods described above. Quantification of RPL19 and 185 mRNA levels was undertaken using the Brilliant SYBR® Green QPCR Master Mix kit (Stratagene, La Jolla, Calif., USA) according to manufacturer's instructions. For each cDNA sample, a singleplex reaction mix was prepared in 0.2 ml microtubes containing forward and reverse primers for 185 or RPL19 at optimized concentrations (Table III), 26 μl of 2× Brilliant® Green QPCR Master Mix, 1.04 ml of diluted cDNA (1:50 for RPL19 and 1:500 dilution for 185) and an appropriate aliquot of Ultra-PureH₂O (Invitrogen) to make a total volume of 52 μl. After thorough vortexing, aliquots of 25 μl was transferred in duplicate to 0.1 ml tubes and capped (Corbett Research, Mortlake, NSW 2137, Australia). The amplification reaction was run on a Rotor-Gene™ 6000 Rotary Analyzer (Corbett Research) using the following conditions: 1 cycle of 95° C. for 10 min; 40 cycles of 95° C. for 15 s and 60° C. for 60 s.

TABLE III
Final concentrations of primers and TaqMan
probes in QPCR reactions for human ALCAM, SFRP2,
NRP1, PR, VCAN, HAS2, FSHR, GLUT4, RPL19 and 18S.

Primers (nM)

| Gene | Forward | Reverse | Taqman probe (nM) |
|---|---|---|---|
| ALCAM | 300 | 200 | 50 |
| SFRP2 | 200 | 300 | 50 |
| NRP1 | 200 | 300 | 100 |
| PR | 300 | 300 | 50 |
| VCAN | 200 | 300 | 50 |
| HAS2 | 300 | 200 | 50 |
| FSHR | 100 | 100 | 50 |
| GLUT4 | 100 | 100 | 100 |
| RPL19 | 100 | 100 | 50 |
| I8S | 300 | 200 | — |

For quantification of candidate gene mRNA levels in individual CC masses, quadruplex (reaction 1: HAS2, FSHR, GLUT4 and RPL19, reaction 2: NRP1, PR, VCAN and RPL19) and triplex (reaction 3: ALCAM, SFRP2 and RPL19) reaction mixes were prepared containing appropriate forward and reverse primers and Taqman probes for candidate genes and the housekeeping gene (RPL19) at optimized concentrations (Table III), 26 μl of 2× Brilliant® Multiplex QPCR Master Mix (Stratagen), neat cDNA and an aliquot of Ultra-Pure H₂O to adjust the total volume to 52 μl. After vortexing, aliquots of 25 μl (including 0.5 μl of neat cDNA) were transferred in duplicate to 0.1 ml tubes and capped (Corbett Research). The amplification reaction was run on a Rotor-Gene™ 6000 Rotary

Analyzer (Corbett Research) using conditions described above.

The relationship between CC numbers and expression levels of RPL19 and 185 was determined by linear regression analysis. The slopes for the lines of best fit for the C_{T} values for 185 using the SYBR green QPCR method, and for RPL19 using the SYBR green and TaqMan QPCR methods, when plotted against the log number of CC, were compared.

Controls included samples that underwent RT-PCR with the exclusion of Superscript III/RNaseout enzyme mix to check the effectiveness of DNase treatment, and reactions that omitted addition of the template. Gene expression levels of HAS2, FSHR, GLUT4, ALCAM, SFRP2, NRP1, PR and VCAN in each sample are presented as fold-changes calculated by the 2^{-ΔΔCt} method (Livak and Schmittgen, 2001) following the correction against a calibrator sample and normalization for RPL19. Serial dilutions (1:1-1:32) of two samples were made in both singleplex and quadruplex reactions to validate PCR efficiency for each gene (≧80% efficiency, where efficiency levels were similar for all three or four genes within each reaction). This included the calculation of the line of best fit (slope±0.1) for target genes and RPL19 mRNA when C_{T} (cycle threshold) values were plotted against log of input RNA or log of total cell number for each CC mass, as well as comparing cycle threshold (C_{T}) values (≦0.4 cycles different) for identical samples for all mRNA transcripts in singleplex and multiplex reactions.

Statistical Analyses

Correlations were analysed using Pearson's R test. Gene expression levels are reported as mean fold change±SEM. Data were log-transformed where necessary and then subjected to either one- or two-way ANOVA using the SPSS statistical package (PASWStatistics 18, IBM, New York, USA). Gene expression levels were also analysed by principal component analysis (PCA) as previously described by Wathlet et al. (2012) using the statistical software R [http://www.r-project.org/ (28 Aug. 2013, date last accessed)].

Mean Gene Expression Levels in Individual CC in Relation to Key Outcomes

With regard to oocyte developmental potential, statistical differences in gene expression levels in CC were investigated for the following parameters:
(i) Oocyte maturity (MII or MI+GV);
(ii) Fertilization rate (2PN or failed fertilization (FF), including abnormal fertilization);
(iii) Good embryonic development on Day 3 (≧7 cell or cell, including FF at fertilization);
(iv) Blastocyst development (good quality blastocyst at Days 5 and 6 (3BB or better; Gardner and Sakkas, 2003) compared with a negative outcome, i.e. embryos that failed to reach blastocyst stage, including those with FF);
(v) Pregnancy rate (positive serum hCG level at Day 14 compared with a negative outcome that included arrested embryos, negative pregnancy and oocytes with FF) and
(vi) Live birth outcome (live birth compared with negative outcomes that included arrested embryos, negative pregnancy and oocytes with FF).

Comparisons between parameters 2 and 6 were made relative to the total number of MIIoocytes available after the denuding step (n=270).

Oocyte Selection by Random Selection

The probability of selecting at least one good quality oocyte (retrospectively assigned by good blastocyst development and pregnancy) from a random selection of one or three COC from the total number of MII oocytes recovered from one woman was calculated according to the formula:

$$P = I - {( \frac{{x}^{neg}}{{x}^{tot}} )}^{n}$$

where P is the probability, x^{neg} is the number of oocytes with a negative outcome, x^{tot} the number of total oocytes collected and n the number of COC selected. From these probability values, the mean probability±SEM of selecting a good quality oocyte was calculated for the 25 women.

Ranking Analysis of Individual MII Oocytes Based on Associated CC Numbers

The technical variability for predicting CC number from RPL19 mRNA levels was calculated by converting the repeated measurement (n=68) of RPL19 mRNA levels in the calibrator sample that was present in every QPCR run to CC number using the equation from the standard curve. Following the calculation of CC numbers from the RPL19 level for each COC, individual MII oocytes within each patient were considered statistically different from each other for associated CC numbers if the number of CC differed by >2×StDev. Individual MII oocytes were then ranked from 1 upwards with 1 indicating the lowest CC number, 2 indicating the second lowest CC number that was significantly different from that of 1, and so on. MII oocytes that showed similar CC numbers with two or more separate ranks (e.g. 1 and 2) were grouped in the lower rank (i.e. 1) to simplify the analysis.

Ranking Analysis of MII Oocytes Based on Associated CC Gene Expression

For individual MII oocytes, 2^{-ΔCT} values were calculated in associated CC for each of the six candidate genes (Livak and Schmittgen, 2001) where the 2^{-ΔCT} values are levels corrected against the housekeeping gene, RPL19. Similarly, the 2×StDev of 2^{-ΔCT} values were calculated for each gene from the repeated measurement of the calibrator sample. The individual values within each patient were considered statistically different from one another if their expression values (2^{-ΔCT}) differed by a value that was >2×StDev of the calibrator sample for each gene. Individual MII oocytes from each woman were then ranked from lowest to highest with respect to CC gene expression levels (2^{-ΔCT}) in CC.

The proportion of individual MII oocytes with different rankings of CC-derived gene expression levels was calculated for each gene within each patient. The mean proportion±SEM of MII oocytes with significantly different expression levels for each of the genes was then determined from associated CC.

Ranking of Individual MII Oocytes for the Selection of Those with Good Blastocyst Development and Live Birth Potential

The MII oocytes from each patient were ranked according to associated CC numbers and CC mRNA expression levels for each of the six candidate genes. These MII oocyte ranks were analysed for each individual parameter, or collectively for groups of parameters, in relation to the outcome of their associated oocyte (good quality blastocyst and live birth) to determine potential indicators of oocyte quality.

To determine the most successful method for selecting at least one good quality oocyte (for a good quality blastocyst or live birth), all selection methods, i.e. (i) using all MII oocytes recovered, (ii) randomly selecting one or three of the recovered MII oocytes and (iii) selecting one or three of the recovered MIIoocytes according to rankings were compared by one- and/or two-way ANOVA (SPSS).

Results

Validation of RPL19 as a Housekeeping Gene

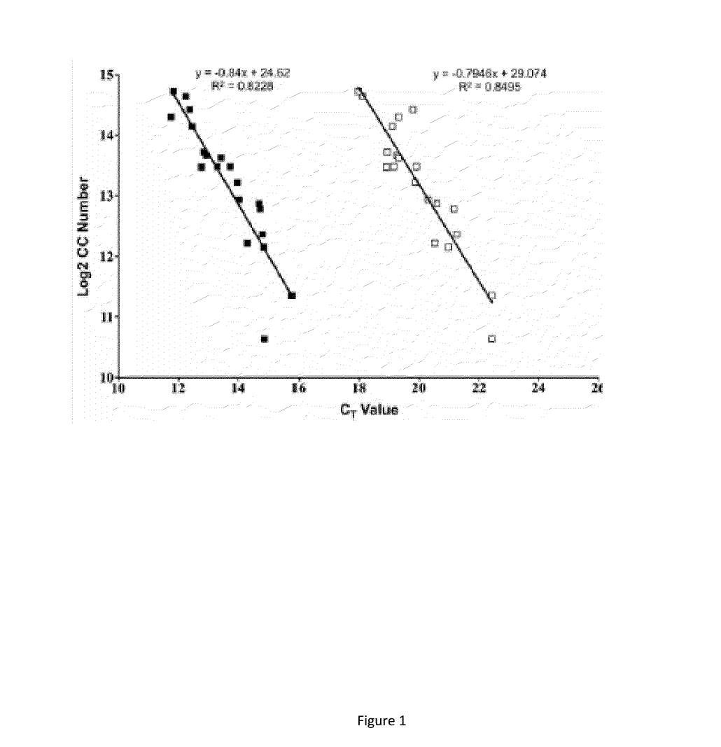

Of the 21 CC masses collected (all associated with MII oocytes) for the validation of the QPCR method, 20 had detectable levels of 185 and RPL19 mRNA. The mean±SEM number of CC counted for these 20 individual CC masses was 19 680±2744 (range 2650-44 900). The relationships between CC number and expression level (a mean value of 4× technical replicates) of RPL19 or 185 are shown in FIG. 1. The slope and correlation efficiency for the lines of best fit were similar for the C_{T} values for 185 using the SYBR green QPCR method and for RPL19 using the TaqMan QPCR method against the log number of CC.

Investigation of Mean CC Numbers in Relation to Embryological and Pregnancy Outcome

Of the 270 CC masses collected in total, 266 (98.5%) had detectable levels of RPL19 mRNA. Using the regression equation of log 2 CC number=−0.7946*(RPL19 C_{T} value)+29.074 derived from FIG. 1, the total number of CC in each CC mass collected was calculated from RPL19 mRNA values. The mean±SEM number of CC calculated for the 266 individual COC was 28 810±1280. The minimum number of CC required to detect RPL19 was estimated to be 66 (C_{T}=28.99). The CC masses that were associated with mature (i.e. MII stage, n=227 from 25 women) oocytes had fewer (P<0.0001) CC compared with those associated with immature (i.e. GV or MI stage, n=31 from 14 women) oocytes (MII+27 606±1156 versus GV+MI=38 489±6518; data from the abnormal oocytes were not included). There were no significant differences in the CC number of COC with regard to fertilization success, D3 cleavage, blastocyst development and positive hCG levels at Day 14 or live birth outcome in comparison with negative outcomes (Table IV).

TABLE IV
Summary of the relationship between CC
numbers and oocyte developmental indicators.

| Indicators | CC numbers (mean ± SEM) | |
|---|---|---|
| All | 28 810 ± 1280 (n = 258) | |
| Oocyte maturity | MII (n = 227) | GV + MI (n = 31) |
| | 27 606^{b} ± 1156 | 38 489^{a} ± 6518 |
| Days 1-6 | | |
| Fertilization | 2PN (n = 183) | FF (n = 44) |
| | 28 420 ± 1338 | 24 202 ± 2083 |
| Day 3 cleavage | ≧7 cell (n = 136) | ≦6 cell (n = 91) |
| | 28 378 ± 1572 | 26 444 ± 1449 |
| Blastocyst | Good quality (n = 52) | NO (n = 175) |
| | 29 510 ± 2069 | 27 030 ± 1369 |
| Pregnancy | | |
| Live birth | LB (n = 19) | NO (n = 175) |
| | 30 852 ± 3950 | 27 030 ± 1369 |

Different letters (a and b) not shared by variables across the rows denote significant difference between CC numbers (mean ± SEM) of oocytes associated with positive and negative outcomes in relation to oocyte developmental indicators (oocyte genomic maturity, P < 0.01; Day 1-6, NS; Pregnancy; NS), FF, failed feritlization, including abnormal fertilization (1PN, 3PN): good quality blastocyst, 3BB or better; NO, negative outcome that included arrested ambryos or those that resulted in failed pregnancies or oocytes that failed to fertilise.

Gene Expression Levels in CCs

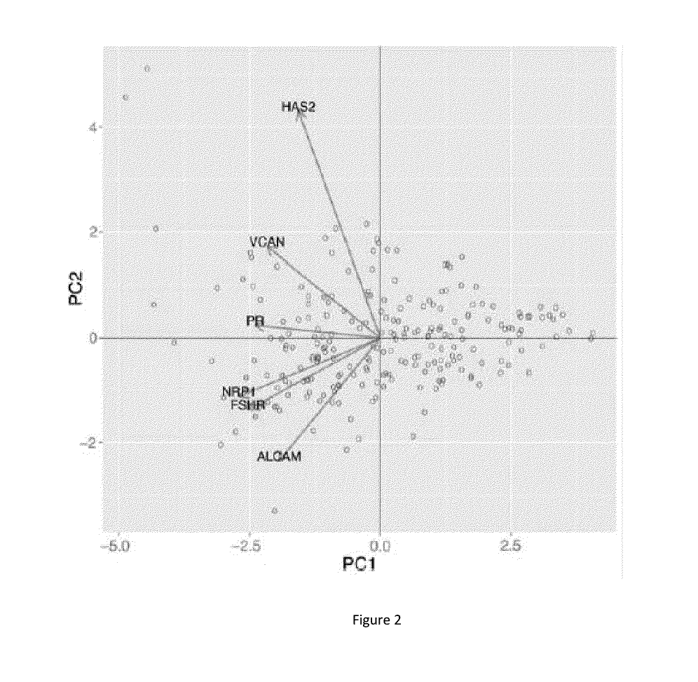

Expression levels of HAS2, FSHR, ALCAM, VCAN, NRP1 and PR mRNA were detectable in 98.5% (266/270) of samples, whilst the proportion of samples with detectable levels of GLUT4 and SFRP2 was low (5.2 and 0.4%, respectively). Overall, the relative expression levels of VCAN mRNA were the highest, followed by those of NRP1, HAS2, ALCAM, FSHR and PR: the statistical differences were VCAN versus NRP1, P<0.0001; NRP1 versus HAS2, P<0.0001; HAS2 versus ALCAM, P<0.05; ALCAM versus FSHR, P<0.0001; and FSHR versus PR, NS). There were highly significant (P<0.0001) linear correlations with respect to the C_{T} values between all six abundant candidate genes in individual CC masses (Table V). The correlation of the expression levels (2̂^{(-ΔΔCT)}) of the six genes was also verified by PCA, where similar results were found. Furthermore, the first two principal components explained 67.7% of the total variance (FIG. 2).

TABLE V
Pearson's R correlation coefficients between
C_{T} values for each gene tested in 266 CC masses.

| Gene | HAS2 | FSHR | ALCAM | VCAN | NRP1 |
|---|---|---|---|---|---|
| FSHR | 0.8501 | | | | |
| ALCAM | 0.8009 | 0.9066 | | | |
| VCAN | 0.8561 | 0.9286 | 0.8996 | | |
| NRP1 | 0.8628 | 0.9554 | 0.9233 | 0.9369 | |
| PR | 0.8928 | 0.9541 | 0.9071 | 0.9301 | 0.9609 |

Gene Expression Levels in Relation to Oocyte Maturity

Mean mRNA levels for VCAN and HAS2 were significantly (VCAN, P<0.0001; HAS2, P<0.05) lower in CC associated with MII oocytes compared with CC associated with MI and GV oocytes. Mean FSHR, ALCAM, NRP1 and PR mRNA levels were not different between CC associated with mature or immature oocytes (Table VI).

TABLE VI
Summary of the relationship between relative expression levels of candidate genes in
CC and oocyte developmental indicators [oocyte maturity, fertilization, development at
Day 3, blastocyst development at Day 5-6 (3BB or better quality) and live births].

| Indicators | HAS2 [AV] | | FSHR [AV] | | ALCAM [AV] | | VCAN [AV] | | NRP [AV] | | PR [AV] | |
|---|---|---|---|---|---|---|---|---|---|---|---|---|
| Oocyte maturity | | | | | | | | | | | | |
| MII | 0.75 ± | 0.3^{b} | 0.85 ± | 0.03 | 0.82 ± | 0.03 | 0.61 ± | 0.02^{b} | 1.00 ± | 0.03 | 0.92 ± | 0.03 |
| MI + GV | 1.00 ± | 0.23^{a} | 1.00 ± | 0.14 | 1.00 ± | 0.08 | 1.00 ± | 0.09^{a} | 0.90 ± | 0.08 | 1.00 ± | 0.11 |
| Fertilization | | | | | | | | | | | | |
| 2PN | 1.00 ± | 0.05 | 0.94 ± | 0.03 | 0.96 ± | 0.04 | 0.95 ± | 0.04 | 0.99 ± | 0.04 | 1.00 ± | 0.03 |
| FF | 0.86 ± | 0.07 | 1.00 ± | 0.06 | 1.00 ± | 0.08 | 1.00 ± | 0.07 | 1.00 ± | 0.08 | 0.97 ± | 0.08 |
| Day 3 | | | | | | | | | | | | |
| ≦7 cell | 1.00 ± | 0.06 | 0.98 ± | 0.04 | 1.00 ± | 0.05 | 1.00 ± | 0.05 | 1.00 ± | 0.04 | 1.00 ± | 0.04 |
| ≧6 cell | 0.89 ± | 0.05 | 1.00 ± | 0.04 | 0.98 ± | 0.06 | 0.95 ± | 0.04 | 0.98 ± | 0.05 | 1.00 ± | 0.05 |
| Day 5-6 | | | | | | | | | | | | |
| Blastocyst | 1.00 ± | 0.10^{a} | 1.00 ± | 0.05 | 1.00 ± | 0.09 | 1.00 ± | 0.06^{a} | 1.00 ± | 0.06 | 1.00 ± | 0.06^{a} |
| NO | 0.78 ± | 0.04^{b} | 0.9 ± | 0.03 | 1.00 ± | 0.04 | 0.79 ± | 0.03^{b} | 0.91 ± | 0.04 | 0.85 ± | 0.03^{b} |
| Live Birth | | | | | | | | | | | | |
| Live Birth | 1.00 ± | 0.18 | 1.00 ± | 0.09 | 1.00 ± | 0.11 | 1.00 ± | 0.10^{a} | 1.00 ± | 0.07 | 1.00 ± | 0.11 |
| NO | 0.78 ± | 0.04 | 0.88 ± | 0.03 | 0.99 ± | 0.04 | 0.80 ± | 0.03^{b} | 0.87 ± | 0.04 | 0.89 ± | 0.03 |

Different letters (i.e. a and b) within genes but between oocyte developmental indicators (i.e. good blastocyts development and live birth outcome) denote significant differences (one- or two-way ANOVA, SPSS) in gene expression levels (mean ± SEM) related to outcome. FF, failed fertilization, including abnormal fertilization (1PN, 3PN): NO, negative outcome that included arrested embryos or those that resulted in failed pregnancies or oocytes that failed to fertilize: AV, arbitrary values.

Gene Expression Levels in Relation to Fertilization or Early Embryonic Development

Mean mRNA levels for all genes were similar in CC associated with oocytes that fertilized normally (2PN) compared with those that either failed to fertilize or were abnormal (i.e. 1PN, 3PN) (Table VI). The mean mRNA levels for all genes were similar in CC associated with oocytes that progressed to embryonic cleavage quickly (≧7 cell on Day 3) compared with those that resulted in slow embryonic cleavage (6 cell on Day 3) and arrested development (Table VI).

Gene Expression Levels in Relation to Blastocyst Development and Live Birth

The mean mRNA levels were significantly higher for HAS2 (P<0.05), VCAN (P<0.005) and PR (P<0.05) in CC associated with oocytes that progressed to a good quality blastocyst compared with CC associated with oocytes that failed to reach a viable blastocyst stage (Table VI). However, the mean levels for FSHR, ALCAM and NRP1 did not differ in CC from oocytes that formed good blastocysts or failed to reach that stage (Table VI).

The mean mRNA levels for VCAN were significantly higher (P<0.05) in CC associated with oocytes that resulted in a healthy term live birth outcome compared with CC associated with a negative outcome. Data from CC associated with oocytes that progressed to good quality blastocysts and were frozen were not included in this comparison. Mean mRNA levels for all other genes were similar in the same comparison (Table VI).

Ranking Analysis of Individual MII Oocytes

Ranking Analysis of Individual MII Oocytes Based on Associated CC Numbers

Of the 232 oocytes that developed to the MII stage of maturation, 227 (9.1±0.7/patient) had measurable RPL19 levels in associated CC from which CC numbers were estimated. Within each woman, 61±3% (5.2±0.3/patient) of MII oocytes differed significantly from each other with respect to the associated CC number (data not shown).

Ranking Analysis of Individual MII Oocytes Based on Expression Levels of a Single Gene in Associated CC

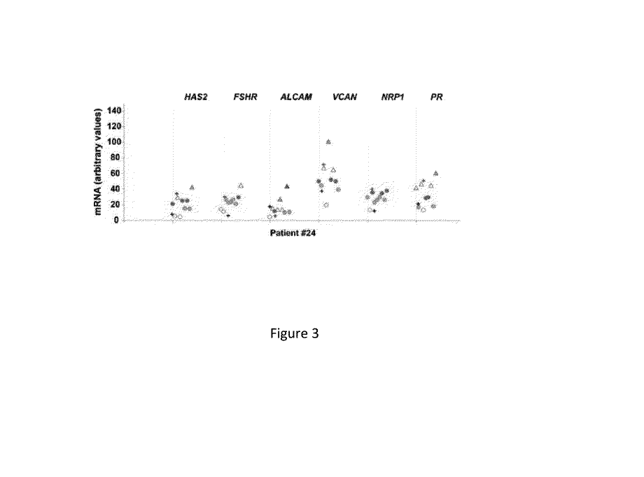

Only CC samples that had measurable levels of RPL19 mRNA and were associated with MII oocytes (n=227) were included in these analyses. Within each woman, the proportion of CC masses with gene expression levels that differed significantly from each other were: 57±3% (5.0±0.4/patient) for HAS2; 55±4% (4.8±0.4/patient) for FSHR; 69±3% (5.9±0.4/patient) for ALCAM; 61±3% (5.2±0.3/patient) for VCAN; 56±3% (5.0±0.4/patient) for NRP1 and 62±4% (5.2±0.4/patient) for PR (e.g. Patient #24, FIG. 3).

Ranking Analysis of Individual MII Oocytes Based on Expression Levels of Six Genes in Associated CC

Only CC samples that had measurable levels of RPL19 mRNA and were associated with MII oocytes (n+227) were included in these analyses. Within each woman, the proportion of CC masses with expression levels that differed significantly for at least one gene was 99.7%. Of the 25 patients, only one woman had two CC masses that shared similar levels of mRNA expression for all six genes. These two individual CC samples had the same number of CC; however, both of the associated MII oocytes had failed in development (e.g. Patient #24, FIG. 3).

Selection of MII Oocytes that Developed to Good Quality Blastocysts

Following the insemination of all 227 MII oocytes, 96% (24/25) of women had at least one oocyte considered to be of good quality (as measured by blastocyst development, i.e. 3BB or better quality on Day 5 or 6 and were transferred or frozen, or Day 3 embryos that were transferred and resulted in a positive pregnancy).

Random Selection

The probability of selecting one good quality oocyte (as assessed by blastocyst development, see above) from each woman at random, from the pool of MII oocytes collected from each woman, was 22.9±2.7%. The probability of at least one good quality oocyte (as assessed by blastocyst development) being included when three MII oocytes are selected for each woman was 50.3±4.4%.

The Use of Ranking Analyses for Oocyte Selection

The combination (sum) of the rankings of four genes (HAS2, FSHR, VCAN and PR) in individual COC provided the highest chance of selecting a single oocyte with good developmental potential (blastocyst development and pregnancy) when the MII oocyte with the highest rankings was selected. There were six cases where the highest-ranking MII oocyte that was associated with a positive outcome shared a similar ranking to another MII oocyte that was associated with a negative outcome. In these instances, differential selection was made between the equally ranked MII oocytes according to firstly the VCAN rank, and if uninformative, then according to the FSHR rank. This method resulted in a 52% (13/25 women) success rate of selecting one good quality oocyte (good quality blastocyst) from the pool of 227 MII oocytes.

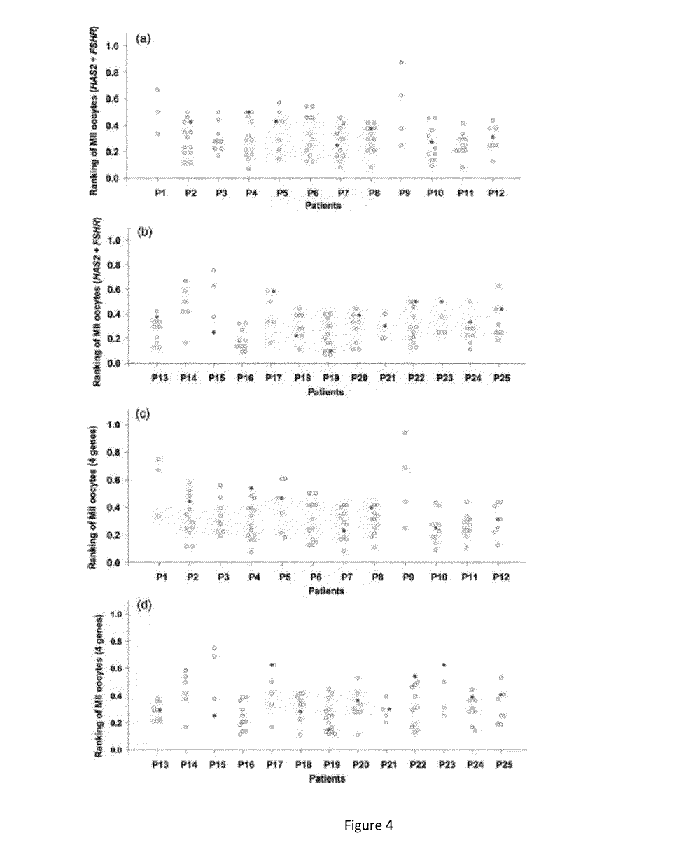

In the case of selecting three MII oocytes with the highest rankings according to four CC genes, the accuracy of selecting at least one oocyte with good developmental potential (good quality blastocyst) was 76% (19/25 women). The combination of the rankings for the two genes, HAS2 and FSHR, also provided high accuracy (80%; 20/25) for selecting at least one good quality oocyte (Table VII and FIG. 4).

TABLE VII
Summary of the percentage (x/25 patients) success
rate in selecting a single oocyte with blastocyst
developmental potential using the ranking of various combinations
of CC-derived expression levels of different candidates
genes and estimated numbers of CC.

| | | Shared | | Shared |
|---|---|---|---|---|
| Ranking | 1 × MII oocyte | rank¹ | 3 × MII oocytes | rank² |
| HAS2 | 40% (10/25) | 4/10 | 80% (20/25) | 4/20 |
| FSHR | 56% (14/25) | 8/14 | 84% (21/25) | 7/21 |
| ALCAM | 28% (7/25) | 3/7 | 60% (15/25) | 4/15 |
| VCAN | 44% (11/25) | 4/11 | 84% (21/25) | 4/21 |
| NRP1 | 40% (10/25) | 5/10 | 72% (18/25) | 4/18 |
| PR | 40% (10/25) | 3/10 | 68% (17/25) | 4/17 |
| CC numbers | 32% (8/25) | 5/8 | 68% (17/25) | 4/17 |
| HAS2 + FSHR | 48% (12/25) | 5/12 | 80% (20/25) | 0/20 |
| HAS2 + FSHR + | 56% (14/25) | 6/14 | 76% (19/25) | 0/19 |
| VCAN + PR | | | | |

The first column represents the parameter that was used for ranking.
The second column (1 × MII oocyte) shows the success rate when selecting a single MII oocyte with the highest ranking value.
The third column (shared rank¹) shows the number of patients where at least two MII oocytes shared the same highest ranking.
The fourth column (3 × MII oocytes) represents the success rate when three MII oocytes are selected according to the three highest rankings.
The fifth column (shared rank²) shows the number of patients where all good quality oocytes within the three highest ranking MII oocytes shared rankings with at least a fourth MII oocytes.

The term “best ranked” as used herein refers to the highest or lowest expression of a single cumulus cell gene or combined expression of more than one cumulus cell gene, or highest or lowest weighted single cumulus cell gene or combined expression of more than one cumulus cell gene.

Oocyte Selection Efficiency for Good Quality Blastocysts

The success rate of recovering at least one good quality oocyte (measured by good blastocyst development) from all MII oocytes collected was 96%. The success rate of including at least one good quality oocyte after selecting a single MII oocyte by the ranking method based on CC gene expression or at random, or by selecting three MII oocytes at random was much lower than if all MII oocytes collected were used (52, 23 and 50%, respectively; P<0.0001). However, when three MII oocytes were selected by the ranking method based on CC gene expression, the success rate was similar to that from using all MII oocytes recovered (80%; P=0.085). Selecting MII oocytes according to the ranking method provided a significantly higher chance of selecting at least one good quality oocyte compared with random selection (1×MII oocyte: 52 versus 23%, P+0.008; 3×MII oocyte: 80 versus 50%, P+0.002).

Selection of Oocytes with Live Birth Potential

The blastocysts that were surplus to requirements were frozen and their live birth outcomes are not known. Thus, to compare the probability of selecting MII oocytes with live birth outcomes using the ranking methods described herein with the potential probability of randomly selecting MII oocytes with live birth outcomes (live birth potential) from all MII oocytes collected (n=227), certain extrapolations were made. Firstly, fresh blastocyst SET live birth rates (42.6%) were used (collected by Fertility Associates in Years 2009-2011 from women<38 years, unpublished data) to calculate the live birth potential of additional blastocysts. Thereafter, the live birth potential of all MII oocytes collected (n=227) was calculated by adding the live birth potential of additional blastocysts to the live birth outcomes of MII oocytes that resulted in a transfer and live birth (19/25 SET).

Random Selection

The probability of selecting one good quality oocyte (measured by live birth potential) at random from each woman was 14.6±1.8% from the pool of 227 MII oocytes across the 25 women. The probability of at least one good quality oocyte (measured by live birth potential) being included when three oocytes were selected from the pool of oocytes collected from each woman was 37.9±4.4%.

The Use of Ranking Analyses for Oocyte Selection

The combination (sum) of the ranking of four genes (HAS2, FSHR, VCAN and PR) in individual COC provided the highest chance of selecting a single oocyte with live birth potential when the MII oocyte with the highest rankings was selected. Further selection was undertaken for the six cases where the highest-ranking MII oocyte shared a similar ranking to another MII oocyte that was associated with a negative outcome using the same rules as described for blastocyst selection. This method resulted in a 30.8±7.5% success rate of picking one MII oocyte with live birth potential from the pool of MII oocytes collected from each woman (n=227 total MII oocytes). In the case of selecting three MII oocytes with the highest rankings according to HAS2 and FSHR, the accuracy of selecting at least one MII oocyte with live birth potential from the pool of 227 MII oocytes was 59.9±9.0%.

Oocyte Selection Efficiency for Live Birth Potential

The live birth rate following SET procedures using all (n=227) MII oocytes undergoing ICSI treatment was 76% (19/25). The live birth potential of a single oocyte after selection by ranking or at random, or of three oocytes after selection at random was calculated to be much lower if all MII oocytes collected were used (31, 15 and 38%, respectively; P<0.0001). However, the live birth potential of three oocytes after selecting from all MII oocytes recovered using the ranking method was similar to the actual live birth rate achieved by using all MII oocytes (60 versus 76%; P=0.206). Selecting MII oocytes according to the ranking method provided a significantly higher chance of selecting at least one good quality oocyte compared with random selection (1×MII oocyte: 31 versus 15%, P<0.05; 3×MII oocytes: 60 versus 38%, P<0.05).

When ranking gene expression of other gene types, it may be that the lowest ranked expression of at least one cumulus cell gene would be appropriate such as cell damage associated genes, where a “best” ranking would correlate lowest (combined) gene expression as a marker for oocyte quality.

### DISCUSSION

The most important finding is that the chance of selecting good quality oocytes recovered from stimulated IVF cycles is significantly improved when CC-derived expression of a selected set of candidate genes were considered, compared with that resulting from random selection. Indeed, the probability of selecting a good quality blastocyst or an oocyte with live birth potential from selecting one (P=0.008 and P<0.05, respectively) or three (P=0.002 and P<0.05, respectively) MII oocytes using the ranking system, compared with random selection, is much improved. Furthermore, we report that the selection of three MII oocytes for each woman, from the total pool of 227 MII oocytes, using the ranking method would have resulted in similar rates for (a) selecting a good quality blastocyst for transfer (80%) or (b) selecting an MII oocyte with live birth potential (60%), compared with classical treatments of using all MII oocytes for treatment (good quality blastocyst: 96%, P=0.085; live birth: 76%, P=0.206, one-way ANOVA). Based on the current retrospective analysis, the ranking method described herein resulted in a higher accuracy of selecting good quality oocytes compared with any other criteria presented so far (Ng et al., 1999; Rienzi et al., 2003; Rienzi et al., 2011) including morphological evaluation of the COC (Ng et al., 1999; Rattanachaiyanont et al., 1999; Lee et al., 2001; Moffatt et al., 2002) and metabolomic profiling of the oocyte culture media (Nel-Themaat and Nagy, 2011). However, when considering the limitations of the method for reliably selecting every good quality oocyte (i.e. at least until the efficiency is further improved), it is suggested that oocytes and/or associated embryos that do not fall within the top three rankings of each patient be cryopreserved, but given a lower priority for future replacement.

This information is highly relevant to countries where oocytes (n=1-3, i.e. until recently in Italy) are randomly selected before ICSI, and 2PN stage embryos are randomly selected at ^{˜}18 h (n=1-3, i.e. Germany, Switzerland; Kufner et al., 2009; Mohler-Kuo et al., 2009; Rienzi et al., 2011). Upon modifications to the methodology described herein, a rapid mRNA analysis system may provide the basis for developing a selection method for MII oocytes (<8 h). If validated, in the current form, this ranking approach could be beneficial for selecting frozen oocytes, especially in countries with a high incidence of SET (e.g. New Zealand, Australia) where embryos are cultured and frozen individually (Macaldowie et al., 2012; Fertility Associates, unpublished results). In those cases when three oocytes/2PN embryos are selected and there are no further legal restrictions regarding the number of embryos replaced/frozen, it is suggested that in order to maximize pregnancy outcomes in SET procedures, the inclusion of further selection criteria such as embryonic development and/or morphology be undertaken.

In countries where no legal or ethical restrictions exist with regard to the numbers of MII oocytes used for ICSI and/or the numbers of embryos cultured to blastocyst stage, this ranking method would offer no significant benefits over blastocyst culture and Day 5 replacement. However, previous studies have reported that CC markers are predictive of live birth outcomes in embryos selected for replacement in an inter-patient analysis and frozen embryos selected for replacement in an intra-patient analysis (lager et al., 2012; Wathlet et al., 2013). The selection models presented in these, and the present, studies offer promise that with additional marker genes and a more rapid screening system, the possibility of earlier predictions and higher efficiencies for selecting good quality oocytes may be realized.

We also report that the mean mRNA levels of HAS2, VCAN and PR were significantly increased in CC associated with oocytes that progressed to good quality blastocysts (3BB or better; Gardner and Sakkas, 2003) compared with that associated with oocytes with failed development. Furthermore, mean mRNA levels of VCAN in CC associated with oocytes that resulted live births were significantly higher compared with that associated with oocytes with failed development. A very important aspect of these findings is that these results are relative to the total number of MII oocytes available after follicular aspiration. Previous studies that showed elevated expression levels of candidate genes in CC associated with blastocyst development and/or pregnancy outcome reported most of their findings for selected groups (i.e. positive and negative groups of embryos selected for transfer) and not relative to total MII oocytes retrieved (Feuerstein et al., 2007; Gebhardt et al., 2011; Wathlet et al., 2011). These and other studies confirmed that VCAN, PTGS2 (Gebhardt et al., 2011) and EFNB2 (Wathlet et al., 2013) levels were higher and SPSB2 (Fragouli et al., 2012) levels tended to be higher in CC associated with transferred embryos that resulted in live births compared with that for failed implantation. Given their association with live birth, we recommend that PTGS2, EFNB2, SPSB2 gene expression be included in future analyses.

The present invention further confirmed that in the case of HAS2, FSHR, VCAN and PR CC-derived gene expression, the key stages of oocyte quality assessment are blastocyst development and live birth outcome. Investigations related to the early stages of embryo development (fertilization and Day 3 embryo development) showed that CC-derived expression levels of these genes are not indicative of positive outcomes for these stages. Previous studies confirmed that mRNA measurements in CC with regard to early embryo development lead to variable and inconsistent results (Gebhardt et al., 2011; Wathlet et al., 2011). Not surprisingly, as recent studies identified, the critical factors in early embryo development appear to be the time and duration of early embryonic cleavage, rather than morphological appearance (Wong et al., 2010).

Analysing mRNA levels in CC can be a suitable method for identifying good candidate genes for oocyte selection. However for successful implementation of this selection method, mRNA levels in CC need to be interrogated for individual COC. Not surprisingly, the highest success rates for the selection of at least one good quality oocyte from the total number of MII oocytes were achieved following the various combinations of rankings of the four genes (HAS2, VCAN, PR and FSHR) that showed higher mean mRNA levels for good blastocyst development and/or pregnancy across all samples. Considering the physiological roles of these genes in important follicular maturation processes such as CC expansion (HAS2 and VCAN; Fulop et al., 1997; Salustri et al., 1999; Russell et al., 2003), follicle development (Otsuka et al., 2005; Caixeta et al., 2009) and ovulation (HAS2 and PR; Park and Mayo, 1991; Gui and Joyce, 2005; Kim et al., 2008; Robker et al., 2009), up-regulation of their expression in CC associated with good quality oocytes is not in itself unexpected.

Despite 43% of the pre-ovulatory follicles present in each woman being of identical diameter on the day of hCG administration, 99.7% of individual COC had significantly different expression levels for at least one of the six candidate genes. Within each woman, the level of heterogeneity between individual CC masses with regard to the expression levels of the genes measured ranged from 56 to 69% and with regard to CC number was 61%. Whilst we acknowledge that the heterogeneity in CC numbers could be an artefact of mechanical disruption or enzymatic perturbation during the recovery process, similar variations in granulosa cell numbers in developing follicles have been reported (McNatty, 1978; McNatty et al., 2010). Thus, few if any antral follicles share an identical endocrine microenvironment, somatic cell composition or responsiveness to gonadotrophins, regardless of size (McNatty, 1978; McNatty and Baird, 1978; McNatty et al., 2010). Hence, these aforementioned findings, together with the very low birth rates (^{˜}7%) from oocytes collected in stimulated IVF cycles (Li et al., 2008; Patrizio and Sakkas, 2009), suggest that ovarian stimulation regimens do not improve the synchrony of follicle development and thus the developmental maturation of the oocytes collected. Particularly, exogenous rFSH treatment does not override the hierarchical pattern of follicular development. The higher success rates obtained in stimulated IVF cycles compared with natural IVF cycles (Fishel et al., 1985; Pelinck et al., 2002) are likely attributed only to the higher number of follicles recruited to a stage where they can be induced to ovulate by exogenous means. These findings support other studies that conclude that current hormonal stimulation protocols are inefficient and alternative follicular maturation methods are required to enhance the yields of viable oocytes (McNatty et al., 2010).

The inventors claim they are the first to validate a multiplex QPCR method capable of the simultaneous measurement of four genes (quadriplex QPCR) in CC masses of individual human COC, offering the advantage over multiple singleplex reactions by reducing the amount of time, reagents and template required (Swango et al., 2007; Hudlow et al., 2008).

An important aspect of the present study was the validation of the housekeeping gene, RPL19. Importantly, this gene was found to be expressed at a similar level to that of many of the target genes measured in this study. The validation of the RPL19 gene was achieved by demonstrating a strong correlation between CC-derived mRNA levels and CC number, as well as comparing expression levels with that of an alternative housekeeping gene, 185. Due to the low-moderate expression levels of the candidate genes, the highly abundant 185 gene was considered less suitable than RPL19 as a housekeeper gene.

The finding that RPL19 mRNA levels were tightly correlated with CC numbers permitted an investigation of the relationship between CC numbers and oocyte developmental indicators. The mean number of CC present in individual COC recovered before IVF treatment was much higher (28 810 c.f. 11 500) compared with that reported previously (Feuerstein et al., 2007). It is reasonable to assume this difference could be attributed to the significantly lower number of COC analysed by the early study (Feuerstein et al., 2007) and/or the inclusion of data from immature oocytes (which have significantly higher number of CC in COC) in the present study. Because the regression equation used in the present study for calculating the total number of CC in each COC was based on data from CC associated with MII oocytes, any data associated with immature oocyte should be interpreted with care. However, the numbers of CC recovered from each oocyte was not predictive of good blastocyst development and/or pregnancy. These data add further evidence to the hypothesis that morphological characteristics of individual COC are poor markers of oocyte quality (Ng et al., 1999; Rattanachaiyanont et al., 1999; Lee et al., 2001; Moffatt et al., 2002; Corn et al., 2005).

### CONCLUSIONS

The main objective of the present study was to determine whether the ranking of MII oocytes based on a cohort of CC-expressed genes would provide a reliable method for selecting good quality MII oocytes from a pool of MII oocytes collected following hormone-stimulated IVF treatments in humans. From these investigations, three major milestones were achieved. First, a multiplex QPCR technique was validated, that was capable of measuring simultaneously the mRNA levels of four genes in CC and permitted an accurate estimation of CC numbers in individual COC. Secondly, the ranking method based on the expression levels of multiple genes in individual CC masses permitted the selection of a good quality oocyte that developed to a good quality blastocyst with 80% efficiency and/or with a live birth potential of 60% per woman by selecting the three highest ranking MII oocytes. This selection method not only provided a significantly better chance of identifying a good quality oocyte compared with random selection, but also resulted in a similar chance compared with using all oocytes available after follicle aspiration. Thirdly, 99.7% of COC retrieved within each woman were significantly different from each other with regard to CC mRNA levels and cell composition. This observation adds further evidence to the findings that ovarian stimulation regimens do not improve developmental synchrony of the follicles recruited and do not override the hierarchical pattern of follicular development.

### Advantages

The present invention offers the notable advantage over the prior art including:
improved accuracy in determining viable oocytes for subsequent implantation and development into a healthy baby;
improved reliability in determining viable oocytes for subsequent implantation and development into a healthy baby; and
a non-invasive method in determining viable oocytes for subsequent implantation and development into a healthy baby.

### Variations

The invention may also be the broadly to consist in the parts, elements and features referred to or indicated in the specification of the application, individually or collectively, in any or all combinations of two or more of the parts, elements or features. Where in the foregoing description reference has been made to integers or components having known equivalents thereof, those integers are herein incorporated as if individually set forth.

It will of course be realised that while the foregoing has been given by way of illustrative example of this invention, all such and other modifications and variations thereto as would be apparent to persons skilled in the art are deemed to fall within the broad scope and ambit of this invention as defined in the appended claims.

Aspects of the present invention have been described by way of example only and it should be appreciated that modifications and additions may be made thereto without departing from the scope thereof as defined in the following claims.

### REFERENCES

Adriaenssens T, Wathlet S, Segers I, Verheyen G, De Vos A, Van der Elst J, Coucke W, Devroey P, Smitz J. Cumulus cell gene expression is associated with oocyte developmental quality and influenced by patient and treatment characteristics. Hum Reprod 2010; 25:1259-1270.
Assidi M, Montag M, Van der Ven K, Sirard M A. Biomarkers of human oocyte developmental competence expressed in cumulus cells before ICSI: a preliminary study. J Assist Reprod Genet 2011; 28:173-188.
Caixeta E S, Ripamonte P, Franco M M, Junior J B, Dode M A. Effect of follicle size on mRNA expression in cumulus cells and oocytes of Bos indicus: an approach to identify marker genes for developmental competence. Reprod Fertil Dev 2009; 21:655-664.
Cillo F, Brevini T A, Antonini S, Paffoni A, Ragni G, Gandolfi F. Association between human oocyte developmental competence and expression levels of some cumulus genes. Reproduction 2007; 134:645-650.
Corn C M, Hauser-Kronberger C, Moser M, Tews G, Ebner T. Predictive value of cumulus cell apoptosis with regard to blastocyst development of corresponding gametes. Fertil Steril 2005; 84:627-633.
Cummins J M, Breen T M, Harrison K L, Shaw J M, Wilson L M, Hennessey J F. A formula for scoring human embryo growth rates in in vitro fertilization: its value in predicting pregnancy and in comparison with visual estimates of embryo quality. J In Vitro Fert Embryo Transf 1986; 3:284-295.
Damario M A, Barmat L, Liu H C, Davis O K, Rosenwaks Z. Dual suppression with oral contraceptives and gonadotrophin releasing-hormone agonists improves in-vitro fertilization outcome in high responder patients. Hum Reprod 1997; 12:2359-2365.
Feuerstein P, Cadoret V, Dalbies-Tran R, Guerif F, Bidault R, Royere D. Gene expression in human cumulus cells: one approach to oocyte competence. Hum Reprod 2007; 22:3069-3077.
Fishel S B, Edwards R G, Purdy J M, Steptoe P C, Webster J, Walters E, Cohen J, Fehilly C, Hewitt J, Rowland G. Implantation, abortion, and birth after in vitro fertilization using the natural menstrual cycle or follicular stimulation with clomiphene citrate and human menopausal gonadotropin. J In Vitro Fert Embryo Transf 1985; 2:123-131.
Fragouli E, Wells D, lager A E, Kayisli U A, Patrizio P. Alteration of gene expression in human cumulus cells as a potential indicator of oocyte aneuploidy. Hum Reprod 2012; 27:2559-2568.
Fujiwara H, Tatsumi K, Kosaka K, Sato Y, Higuchi T, Yoshioka S, Maeda M, Ueda M, Fujii S. Human blastocysts and endometrial epithelial cells express activated leukocyte cell adhesion molecule (ALCAM/CD166). J Clin Endocrinol Metab 2003; 88:3437-3443.
Fulop C, Salustri A, Hascall V C. Coding sequence of a hyaluronan synthase homologue expressed during expansion of the mouse cumulus-oocyte complex. Arch Biochem Biophys 1997; 337:261-266.
Gardner D K, Sakkas D. Assessment of embryo viability: the ability to select a single embryo for transfer—a review. Placenta 2003; 24(Suppl. B): S5-S12.
Gebhardt K M, Feil D K, Dunning K R, Lane M, Russell D L. Human cumulus cell gene expression as a biomarker of pregnancy outcome after single embryo transfer. Fertil Steril 2011; 96:47-52, e42.
Gui L M, Joyce I M. RNA interference evidence that growth differentiation factor-9 mediates oocyte regulation of cumulus expansion in mice. Biol Reprod 2005; 72:195-199.
Hernandez-Gonzalez I, Gonzalez-Robayna I, Shimada M, Wayne C M, Ochsner S A, White L, Richards J S. Gene expression profiles of cumulus cell oocyte complexes during ovulation reveal cumulus cells express neuronal and immune-related genes: does this expand their role in the ovulation process? Mol Endocrinol 2006; 20:1300-1321.
Hudlow W R, Chong M D, Swango K L, Timken M D, Buoncristiani M R. A quadruplex real-time qPCR assay for the simultaneous assessment of total human DNA, human male DNA, DNA degradation and the presence of PCR inhibitors in forensic samples: a diagnostic tool for STR typing. Forensic Sci Int Genet 2008; 2:108-125.
lager A E, Kocabas A M, Out H H, Ruppel P, Langerveld A, Schnarr P, Suarez M, Jarrett J C, Conaghan J, Rosa G J et al. Identification of a novel gene set in human cumulus cells predictive of an oocyte's pregnancy potential. Fertil Steril 2012; 99:745-752, e6.
Kim J, Sato M, Li Q, Lydon J P, Demayo F J, Bagchi I C, Bagchi M K. Peroxisome proliferator-activated receptor gammais a target of progesterone regulation in the preovulatory follicles and controlsovulation in mice. Mol Cell Biol 2008; 28:1770-1782.
Kufner K, Tonne M, Barth J. What is to be done with surplus embryos? Attitude formation with ambivalence in German fertility patients. Reprod Biomed Online 2009; 18(Suppl. 1):68-77.
Land J A, Evers J L. Risks and complications in assisted reproduction techniques: Report of an ESHRE consensus meeting. Hum Reprod 2003; 18:455-457.
Lee K S, Joo B S, Na Y J, Yoon M S, Choi O H, Kim W W. Cumulus cells apoptosis as an indicator to predict the quality of oocytes and the outcome of IVF-ET. J Assist Reprod Genet 2001; 18:490-498.
Li Q, McKenzie U, Matzuk M M. Revisiting oocyte-somatic cell interactions: in search of novel intrafollicular predictors and regulators of oocyte developmental competence. Mol Hum Reprod 2008; 14:673-678.
Livak K J, Schmittgen T D. Analysis of relative gene expression data using real-time quantitative PCR and the 2(−Delta Delta C(T)) Method. Methods 2001; 25:402-408.
Macaldowie A, Wang Y A, Chambers G M, Sullivan E A. Assisted Reproduction Technology in Australia and New Zealand 2010. Assisted Reproduction Technology Series No. 16. Cat. No. PER 55. Canberra: AIHW, 2012.
McKenzie U, Pangas S A, Carson S A, Kovanci E, Cisneros P, Buster J E, Amato P, Matzuk M M. Human cumulus granulosa cell gene expression: a predictor of fertilization and embryo selection in women undergoing IVF. Hum Reprod 2004; 19:2869-2874.
McNatty K P. Cyclic changes in antral fluid hormone concentrations in humans. Clin Endocrinol Metab 1978; 7:577-600.
McNatty K P, Baird D T. Relationship between follicle-stimulating hormone, androstenedione and oestradiol in human follicular fluid. J Endocrinol 1978; 76:527-531.
McNatty K P, Heath D A, Hudson N L, Reader K L, Quirke L, Lun S, Juengel J L. The conflict between hierarchical ovarian follicular development and superovulation treatment. Reproduction 2010; 140:287-294.
Moffatt O, Drury S, Tomlinson M, Afnan M, Sakkas D. The apoptotic profile of human cumulus cells changes with patient age and after exposure to sperm but not in relation to oocyte maturity. Fertil Steril 2002; 77:1006-1011.
Mohler-Kuo M, Zellweger U, Duran A, Hohl M K, Gutzwiller F, Mutsch M. Attitudes of couples towards the destination of surplus embryos: results among couples with cryopreserved embryos in Switzerland. Hum Reprod 2009; 24:1930-1938.
Nel-Themaat L, Nagy Z P. A review of the promises and pitfalls of oocyte and embryo metabolomics. Placenta 2011; 32(Suppl. 3):5257-5263. Ng S T, Chang T H, Wu T C. Prediction of the rates of fertilization, cleavage, and pregnancy success by cumulus-coronal morphology in an in vitro fertilization program. Fertil Steril 1999; 72:412-417.
Otsuka F, Moore R K, Wang X, Sharma S, Miyoshi T, Shimasaki S. Essential role of the oocyte in estrogen amplification of follicle-stimulating hormone signaling in granulosa cells. Endocrinology 2005; 146:3362-3367.
Park O K, Mayo K E. Transient expression of progesterone receptor messenger RNA in ovarian granulosa cells after the preovulatory luteinizing hormone surge. Mol Endocrinol 1991; 5:967-978.
Patrizio P, Sakkas D. From oocyte to baby: a clinical evaluation of the biological efficiency of in vitro fertilization. Fertil Steril 2009; 91:1061-1066.
Pelinck M J, Hoek A, Simons A H, Heineman M J. Efficacy of natural cycle IVF: a review of the literature. Hum Reprod Update 2002; 8:129-139.
Rattanachaiyanont M, Leader A, Leveille M C. Lack of correlation between oocyte-corona-cumulus complex morphology and nuclear maturity of oocytes collected in stimulated cycles for intracytoplasmic sperm injection. Fertil Steril 1999; 71:937-940.
Rienzi L, Ubaldi F, Martinez F, lacobelli M, Minasi M G, Ferrero S, Tesarik J, Greco E. Relationship between meiotic spindle location with regard to the polar body position and oocyte developmental potential after ICSI. Hum Reprod 2003; 18:1289-1293.
Rienzi L, Vajta G, Ubaldi F. Predictive value of oocyte morphology in human IVF: a systematic review of the literature. Hum Reprod Update 2011; 17:34-45.
Roberts R, Stark J, Iatropoulou A, Becker D L, Franks S, Hardy K. Energy substrate metabolism of mouse cumulus-oocyte complexes: response to follicle-stimulating hormone is mediated by the phosphatidylinositol 3-kinase pathway and is associated with oocyte maturation. Biol Reprod 2004; 71:199-209.
Robker R L, Akison L K, Russell D L. Control of oocyte release by progesterone receptor-regulated gene expression. Nucl Recept Signal 2009; 7:e012.
Russell D L, Ochsner S A, Hsieh M, Mulders S, Richards J S. Hormone regulated expression and localization of versican in the rodent ovary. Endocrinology 2003; 144:1020-1031.
Sakkas D, Shoukir Y, Chardonnens D, Bianchi P G, Campana A. Early cleavage of human embryos to the two-cell stage after intracytoplasmic sperm injection as an indicator of embryo viability. Hum Reprod 1998; 13:182-187.
Salustri A, Camaioni A, Di Giacomo M, Fulop C, Hascall V C. Hyaluronan and proteoglycans in ovarian follicles. Hum Reprod Update 1999; 5:293-301.
Swango K L, Hudlow W R, Timken M D, Buoncristiani M R. Developmental validation of a multiplex qPCR assay for assessing the quantity and quality of nuclear DNA in forensic samples. Forensic Sci Int 2007; 170: 35-45.
Wathlet S, Adriaenssens T, Segers I, Verheyen G, Van de Velde H, Coucke W, Ron El R, Devroey P, Smitz J. Cumulus cell gene expression predicts better cleavage-stage embryo or blastocyst development and pregnancy for ICSI patients. Hum Reprod 2011; 26:1035-1051.
Wathlet S, Adriaenssens T, Segers I, Verheyen G, Janssens R, Coucke W, Devroey P, Smitz J. New candidate genes to predict pregnancy outcome in single embryo transfer cycles when using cumulus cell gene expression. Fertil Steril 2012; 98:432-439, e431-434.
Wathlet S, Adriaenssens T, Segers I, Verheyen G, Van Landuyt L, Coucke W, Devroey P, Smitz J. Pregnancy prediction in single embryo transfer cycles after ICSI using QPCR: validation in oocytes from the same cohort. PLoS One 2013; 8(4, e54226):1-10.
Wong C C, Loewke K E, Bossert N L, Behr B, De Jonge O, Baer T M, Reijo Pera R A. Non-invasive imaging of human embryos before embryonic genome activation predicts development to the blastocyst stage. Nat Biotechnol 2010; 28:1115-1121.

1. An in-vitro method of selection of viable oocytes from a population of mammalian oocytes from an individual mammal comprising the steps:
a) measuring the level of expression of at least one cumulus cell gene in cumulus cells associated with each of the population of mammalian oocytes as a marker for oocyte quality;
b) ranking each oocyte from the population of mammalian oocytes by its measured gene expression from step a); and
c) selecting a subset of viable mammalian oocytes that have the best ranked gene expression from step b) for subsequent in vitro fertilisation.

2. The in-vitro method of selection of viable oocytes of claim 1 wherein the measurement step a) is carried out at a ‘MII’ stage of oocyte development.

3. The in-vitro method of selection of viable oocytes of claim 1, wherein the mammalian oocytes are human oocytes.

4. The in-vitro method of selection of viable oocytes of claim 1, wherein the at least one cumulus cell gene is between 4 and 32 cumulus cell genes.

5. The in-vitro method of selection of viable oocytes of claim 4, wherein the at least one cumulus cell gene is 4 cumulus cell genes.

6. The in-vitro method of selection of viable oocytes of claim 1 wherein the step b) is ranking each oocyte from the population of mammalian oocytes by its combined measured gene expression and step c) is selecting a subset of viable mammalian oocytes that have the best combined ranked gene expression.

7. The in-vitro method of selection of viable oocytes of claim 1 wherein the at least one cumulus cell gene is a follicular maturation process gene.

8. The in-vitro method of selection of viable oocytes of claim 1 wherein the cumulus cell gene is selected from the group comprising: versican (VCAN); follicle-stimulating hormone receptor (FSHR); hyaluronan synthase 2 (HAS2); progesterone receptor (PR); neuropilin 1 (NRP1); activated leukocyte cell adhesion molecule (ALCAM).

9. The in-vitro method of selection of viable oocytes of claim 1, wherein the level of expression of the at least one cumulus cell genes in step a) is measured by quantitative Polymerase Chain Reaction (qPCR).

10. The in-vitro method of selection of viable oocytes of claim 9, wherein the qPCR is multiplex PCR.

11. The in-vitro method of selection of viable oocytes of claim 9, wherein the levels of expression of the at least one cumulus cell genes in step a) as measured by quantitative Polymerase Chain Reaction (qPCR) is completed within 8 to 24 hours.

12. The in-vitro method of selection of viable oocytes of claim 1, wherein the subset of viable mammalian oocytes selected in step c) is three.

13. An in-vitro method of assisted reproduction comprising the steps:
a) measuring the level of expression of at least one cumulus cell gene in the cumulus cells associated with each of the population of mammalian oocytes from an individual mammal as a marker for oocyte quality;
b) ranking each oocyte from the population of mammalian oocytes by its measured gene expression from step a); and
c) selecting a subset of viable mammalian oocytes that have the best ranked gene expression from step b) for subsequent in vitro fertilisation.

14. A kitset for carrying out an in-vitro method of selection of viable oocytes from a population of mammalian oocytes from an individual mammal, the kitset comprising at least one pair of PCR primers configured to bind to a cumulus cell gene, the method comprising the steps:
a) measuring the level of expression of at least one cumulus cell gene in the cumulus cells associated with each of the population of mammalian oocytes as a maker for oocyte quality;
b) ranking each oocyte from the population of mammalian oocytes by its measured gene expression from step a); and
c) selecting a subset of viable mammalian oocytes that have the best ranked gene expression from step b) for subsequent in vitro fertilisation.

15. A method of detecting potentially viable oocytes from a population of mammalian oocytes from an individual comprising the step of measuring the level of expression of at least one cumulus cell gene in the cumulus cells associated with each of the population of mammalian oocytes as a marker for oocyte quality.
